(19) United States
(12) Patent Application Publication
(10) Pub. No.: US 2026/0341660 A1
(43) Pub. Date: Oct. 29, 2026

(54) Mass Spectrometry Detection of Chemical Threats after Ambient Desorption/Ionization with Adducting Agents

(71) Applicant: Advion, Inc., Ithaca, NY (US)

(72) Inventors: Changtong Hao, Ithaca, NY (US); Daniel Eikel, Lansing, NY (US); Jimmie Oxley, Narraganset, RI (US); Michael Chapman, Guilford, CT (US)

(73) Assignee: Advion, Inc., Ithaca, NY (US)

(21) Appl. No.: 19/468,177

(22) Filed: Feb. 3, 2026

Related U.S. Application Data
(60) Provisional application No. 63/753,192, filed on Feb. 3, 2025.

(51) Int. Cl. H01J 49/04; G01N 33/22; H01J 49/00; H01J 49/16

(52) U.S. Cl. CPC H01J 49/0431; G01N 33/227; H01J 49/0031; H01J 49/165

(57) ABSTRACT

The inventors recognize that chemical threats such as explosives can be difficult to detect using conventional testing methods such as mass spectrometry, especially at low concentrations. The inventors further recognize that adducts, while usually considered undesirable and unwanted by-products, can be easy to detect, can improve sensitivity of substances even in trace amounts, and can reduce fragmentation in energetic molecules, making the intentional creation of adducts a sensitive and viable approach for mass spectrometry detection of such molecules under electrospray ionization, for example.

### FIELD OF THE INVENTION

The present disclosure is directed to an adducting agent, and more specifically to a method of using an adducting agent for the intentional formation of molecule adducts to detect chemical threats using mass spectrometry.

### BACKGROUND

In mass spectrometry, chemicals are detected by the mass-to-charge ratio of their respective ions formed during the desorption and ionization of the chemical into or in the gas phase. Various ionization techniques exist, for example electron impact (EI), electrospray ionization (ESI), matrix assisted desorption ionization (MALDI), or atmospheric pressure chemical ionization (APCI). All techniques have in common the desire to bring one chemical molecule from a solid or liquid state into the gas phase and to create ideally one and the same ion of each molecule. Usually, this ion is the entire molecule as a whole (not a fragment) and is ionized as a radical ion, a molecular ion with a proton attached (M+H)+ or subtracted (M−H)−, or multiple charges attached/detached for a multiply charged species of the molecular ion.

Fragment ions, as they form in the desorption/ionization process, are usually undesired since they reduce signal intensity at the molecular ion mass-to-charge channel. However, fragment ions can be induced intentionally (either in source or in the mass spectrometer itself) and can then help provide characteristic ions that identify a chemical unequivocally.

Ions created in the desorption/ionization process can also be adducts of the molecular ion. These are usually considered unwanted by-products of salt contamination such as sodium (M+Na)+ or potassium (M+K)+ but can also be intentionally formed with adducting agents such as formate or acetate (see S Mathias, M Amerio-Cox, T Jackson, D Douce, A Sage, P Luke, R Sleeman, C Crean and Patrick Sears: Selectivity of Explosives Analysis with Ambient Ionization Single Quadrupole Mass Spectrometry: Implications for Trace Detection. J. Am. Soc. Mass Spectrom. 2024, 35, 50-61 and D Burns, S Mathias, BJ McCullough, CJ Hopley, D Douce, N Lumley, S Bajic and P Sears: Ambient ionisation mass spectrometry for the trace detection of explosives using a portable mass spectrometer. International Journal of Mass Spectrometry 471 (2022) 116735).

Adducts usually form as an incomplete reaction with molecules present in the liquid or solid state of the sample. Because their reaction is typically incomplete, adduct formation is causing a reduction in mass spectrometry sensitivity (similar to fragmentation) since only a portion of the molecules form adducts, whereas a higher number will remain a protonated/de-protonated molecular ion. As such, adduct ions are usually considered undesirable in mass spectrometry and great effort is extended on the sample preparation before mass spectrometry detection to reduce salt load and avoid the formation of them.

Adduct ions can be formed in various ionization techniques including direct and ambient ionization techniques; however, some ionization is less susceptible to adduct formation, for example APCI, whereas others are more susceptible to it, for example ESI. In general, the softer the desorption/ionization, the more prone to adduct formation the technique is. Softness in ionization is characterized by a limited amount of energy transferred to the molecule during the desorption/ionization process; this limited energy transfer allows the adduct to stay with the molecule during the process. However, the soft ionization of ESI has significant advantages for otherwise energetically labile molecules and has become a gold standard of desorption/ionization in mass spectrometry coupled with either liquid chromatographic separation (HPLC) or in applications with ambient direct sample introduction.

To the extent that specific patents/publications/products are discussed in this disclosure, these discussions should not be taken as an admission that the discussed patents/publications/products are prior art for patent law purposes. For example, some or all of the discussed patents/publications/products may not be sufficiently early in time, may not reflect subject matter developed early enough in time and/or may not be sufficiently enabling so as to amount to prior art for patent law purposes. To the extent that specific patents/publications/products are discussed throughout the application, the descriptions/disclosures of which are all hereby incorporated by reference into this document in their respective entirety(ies).

### SUMMARY

The inventors recognize that chemical threats such as explosives can be difficult to detect using conventional testing methods such as mass spectrometry, especially at low concentrations. The inventors further recognize that adducts, while usually considered undesirable and unwanted by-products, can be easy to detect, can improve sensitivity of substances even in trace amounts, and can reduce fragmentation in energetic molecules, making the intentional creation of adducts a sensitive and viable approach for mass spectrometry (MS) detection of such molecules under electrospray ionization, for example.

It is therefore a principal object and advantage of embodiments of the present disclosure to provide a novel adducting agent for the intentional formation of molecule adducts in mass spectrometry detection of chemical threats such as explosives and a method of using the adducting agent to detect the same.

In one embodiment, a method of detecting a target analyte by mass spectrometry is described. The method can comprise the steps of 1) contacting a sample suspected of containing the target analyte with a solution comprising Bromo-difluoro-acetate anion (BrCF₂COO—) in an electrospray-compatible solvent; 2) subjecting the contacted sample to negative-mode electrospray ionization to generate gas-phase ions; and 3) detecting an adduct ion comprising the target analyte and Bromo-difluoro-acetate, wherein the detected adduct ion exhibits a bromine isotopic doublet having peaks separated by about 2 Daltons and an intensity ratio of about 1:1, and wherein the mass-to-charge ratio of the adduct ion is shifted relative to the mass-to-charge ratio of the target analyte or, when present, the deprotonated molecular ion of the target analyte.

In some embodiments, the solution comprises Bromo-difluoro-acetate at a concentration of about 10 mol/L.

In some embodiments, the electrospray-compatible solvent comprises methanol.

In some embodiments, the electrospray-compatible solvent comprises ethanol.

In some embodiments, the target analyte is selected from PETN, RD, and nitroglycerin.

In accordance with another embodiment, a method of detecting a chemical threat present on a surface is described. The method can comprise the steps of: 1) applying a solution comprising an adducting agent to the surface under conditions effective to form, on the surface or in the applied solution, an adduct between the chemical threat and the adducting agent; 2) transferring at least a portion of the applied solution to an electrospray emitter and applying a capillary voltage to generate gas-phase ions comprising ions of the adduct; and 3) analyzing the ions with a mass spectrometer to detect the chemical threat; wherein the adduct has the structure of Formula (I):

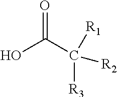

wherein R1 is selected from H, X, and CX3; wherein R2 is selected from H, F, and X; wherein R3 is selected from H, F, and X; and wherein X is selected Cl, Br, and I.

In some embodiments, the solution comprises the adducting agent at a concentration of about 10 mol/L.

In some embodiments, the chemical threat is selected from PETN, RD, and nitroglycerin.

In accordance with another embodiment, a method of detecting a target analyte by mass spectrometry is described. The method can comprise the steps of: 1) contacting a sample suspected of containing the target analyte with a solution comprising an anionic halogenated carboxylate containing one or more halogen atoms selected from bromine and chlorine; 2) generating negative-mode electrospray ions; 3) subjecting the contacted sample to negative-mode electrospray ionization to generate gas-phase ions; and 4) detecting an adduct ion comprising the target analyte and the halogenated carboxylate, wherein the detected adduct ion exhibits a halogen isotopic pattern characteristic of the one or more halogen atoms present, and wherein the mass-to-charge ratio of the adduct ion is shifted relative to the mass-to-charge ratio of the target analyte or, when present, the deprotonated molecular ion of the target analyte.

In some embodiments, the halogenated carboxylate is Bromo-difluoro-acetate.

In some embodiments, the halogenated carboxylate comprises bromine and the detected adduct ion exhibits a bromine isotopic doublet with peaks separated by about 2 Daltons and an intensity ratio of 1:1.

In some embodiments, the halogenated carboxylate comprises chlorine and the detected adduct ion exhibits a chlorine isotopic doublet with peaks separated by about 2 Daltons and an intensity ratio of 3:1.

In some embodiments, detecting comprises selected ion monitoring or multiple reaction monitoring of m/z values corresponding to the adduct ion and its isotopologues.

In some embodiments, the solution comprises the anionic halogenated carboxylate at a concentration of about 10 mol/L.

In some embodiments, the target analyte is selected from PETN, RD, and nitroglycerin.

These and other aspects of the invention will be apparent from the embodiments described below.

### BRIEF DESCRIPTION OF THE DRAWINGS

The present invention will be more fully understood and appreciated by reading the following Detailed Description in conjunction with the accompanying drawings, in which:

FIG. 1 is a chemical representation of a Bromo-difluoro-acetate molecule.

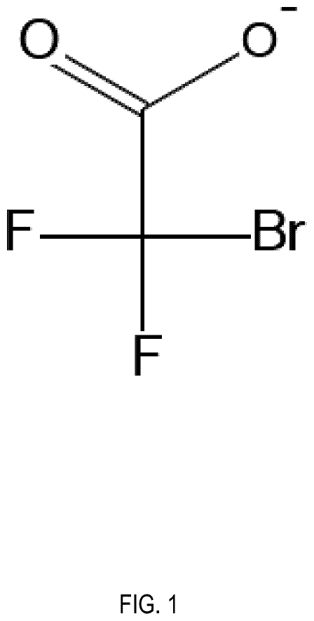

FIG. 2 is a chemical representation of an example genus description of a novel adducting agent, according to an aspect of the present disclosure.

FIG. 3 is a representation of three ambient desorption/ionization mass spectra results of RDX, according to an aspect of the present disclosure.

FIG. 4 is a representation of three ambient desorption/ionization mass spectra results of PETN, according to an aspect of the present disclosure.

FIG. 5 is chart depicting threat molecules and the respective adduct ions formed in electrospray ionization, according to an aspect of the present disclosure.

### DETAILED DESCRIPTION OF EMBODIMENTS

Aspects of the present embodiments and certain features, advantages, and details thereof, are explained more fully below with reference to the non-limiting examples illustrated in the accompanying drawings. Descriptions of well-known structures are omitted so as not to unnecessarily obscure the invention in detail. It should be understood, however, that the detailed description and the specific non-limiting examples, while indicating aspects of the invention, are given by way of illustration only, and are not by way of limitation. Various substitutions, modifications, additions, and/or arrangements, within the spirit and/or scope of the underlying inventive concepts will be apparent to those skilled in the art from this disclosure.

While embodiments of the present invention have been particularly shown and described with reference to certain exemplary embodiments, it will be understood by one skilled in the art that various changes in detail may be affected therein without departing from the spirit and scope of the invention as defined by claims that can be supported by the written description and drawings. Further, where exemplary embodiments are described with reference to a certain number of elements, it will be understood that the exemplary embodiments can be practiced utilizing either less than or more than the certain number of elements. If elements shown in a particular Figure discussed below are not specifically identified with respect to that Figure, the elements should be sufficiently identified with respect to at least one other Figure.

The present disclosure describes a novel adducting agent for the intentional formation of molecule adducts in mass spectrometry detection of chemical threats such as explosives. Bromo-difluoro-acetate was found to be a good adducting agent in ESI-MS because of its properties. For instance, first, it contains bromine which gives it a strong, unmistakable isotope signature. As bromine naturally has two isotopes, it produces two peaks 2 m/z apart with nearly 1:1 intensity. Thus, Bromo-difluoro-acetate adducts can be highly specific, hard to confuse with background ions, and easy to detect even on low-resolution MS instruments. Second, it forms stable, clean negative-mode adducts. Since it is an anion and a carboxylate, it can form strong, predictable adducts. Third, it is electrospray friendly, meaning it readily participates in ESI droplet chemistry.

High energetic molecules benefit from soft ionization such as ESI and can form a significant degree of adducts, and this disclosure recognizes that the intentional creation of the adduct molecule is a sensitive and viable approach for mass spectrometric detection of such molecules under ESI conditions. A molecule adduct can have significant advantages to be detected in MS compared to the simple protonated/de-protonated molecule in that the adducting agent can carry a halogen atom. This can create two or more isotopic signals in the mass spectrometer which allows for a more specific detection of the threat molecule in two (or more) distinct m/z channels, separated by a sufficient amount of m/z space to be resolved even with cost-effective low-resolution mass spectrometry (as should be understood by one of ordinary skill in the art in conjunction with a review of this disclosure).

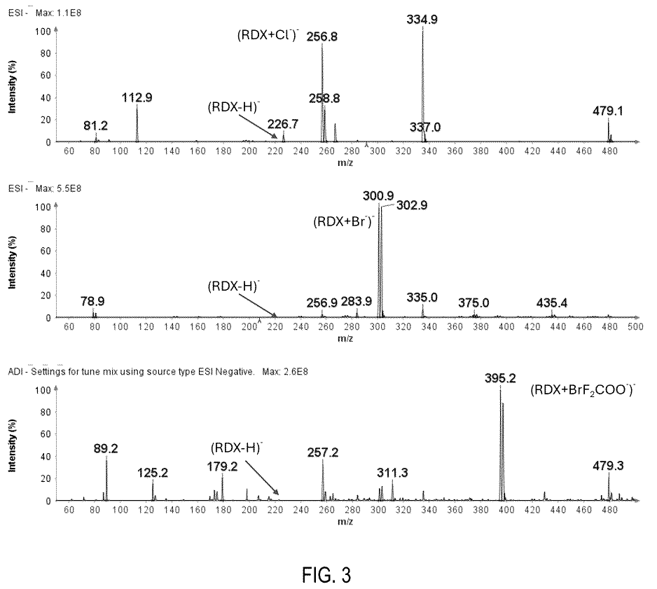

For example, a chlorine adduct of the explosive PETN (Pentaerythritol tetranitrate) can be formed. It shows a mass spectrometry signal at m/z 351.0 and 353.0 compared to the de-protonated species which shows only a single signal in the detection channel at m/z 315.0 for the (M−H)− ion (see FIG. 3). In the case of Chlorine as an adducting agent, the isotope distribution found depends on the natural isotope distribution of chlorine itself (independent of the threat detected) and is roughly 3 to 1 for 35Cl to 37Cl (at the respective total ion mass of m/z 351.0 and 353.0). This allows for a specific pattern recognition of MS signals obtained, but makes the second m/z channel significantly less intense than the first and limits the use of the second channel as a threat detection channel. At very low concentrations of the threat, the minor signal would disappear in the background noise of the mass spectrometer well before the major signal and thereby not improve the assay specificity for such an adduct at very low concentrations.

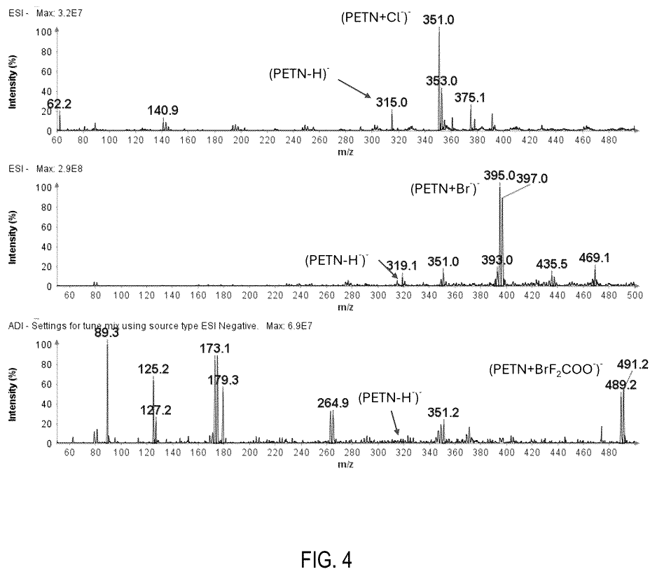

A better choice of adduct can be a molecule with a bromine atom. Natural abundance of bromine can create two almost equal isotopic signals and make two mass spectrometric channels for each underlying threat molecule (as should be understood by a person of ordinary skill in conjunction with a review of this disclosure). In other words, the doublet pattern can be very recognizable, as its shape can be easily identifiable and thus hard to confuse with background ions. In the case of bromine, even at lower concentrations of the threat present, both signals (i.e., bromine isotopic doublet) can show near equal sensitivity, and both can be used for the threat detection decision in addition to its very prominent signal pattern (see, e.g., FIG. 4 showing PETN with a bromine adduct to provide two signals as m/z 395.0 and 397.0 at near equal intensity).

A second benefit of adducting agents, and their use in threat detection, is the mass shift that the adduct causes compared to the protonated or de-protonated molecule alone. Other chemicals interfering with the mass spectrometry detection (general chemical background noise of the analysis) are not equally distributed over the mass-to-charge range but rather taper off beyond m/z 300 (as should be understood by a person of ordinary skill in the art in conjunction with this disclosure). Any adducting agent that can increase the mass-to-charge ratio of the molecule under investigation has therefore the potential to improve the sensitivity of the assay since the general chemical background noise present at the new mass-to-charge channel will be lower.

And lastly, some chemicals cannot be desorbed/ionized by themselves, even employing a soft ionization such as electrospray. For these compounds, an adduct is the only way to detect the chemical with ESI (e.g., RDX (1,3,5-Trinitroperhydro-1,3,5-triazine).

With regards to the adduct formation, an adducting agent can be intentionally designed to carry a bromine atom (for benefits described above) and also show enhanced abilities to stabilize the ionic bond with the threat molecule, for example by adding electronegative pull that will enhance the distribution of electronegativity across multiple atoms in the molecule. A near equal isotopic distribution of 1 to 1 can also be achieved with three atoms of Chlorine in the adducting agent or three atoms of bromine and multiples of these numbers. In a preferred embodiment, the molecule that was found to be very suitable for the purposes described above is Bromo-difluoro-acetate (see FIGS. 3-4), where the additional fluorine atoms improve electronegative pull on the binding site of the oxygen acetate group and thereby stabilize the anion from the complex formed with the target molecule. This enhances the reaction towards a near complete adduct formation and improves desorption/ionization efficiency. The overall mass of the Bromo-difluoro-acetate adduct is 173 amu higher (79Br) than the comparable deprotonated molecule and provides a significant shift in the m/z range the molecule can now be detected, a mass shift improvement over the other possible adduct agents such as chlorine (+35 amu with the 35Cl isotope), bromine (+79 amu with the 79Br isotope), or acetate alone (+59 amu, data not shown).

As shown in FIG. 3, three ambient desorption/ionization mass spectra in negative ion mode ESI (A, B and C) of RDX using: A) Chlorine as an adducting agent at 50 μmol/L; B) Bromine as an adducting agent at 20 μmol/L, and C) Bromo-difluoro-acetate as an adducting agent at 10 μmol/L in solution are shown. Theoretical location of the molecular ion was indicated at m z 221 for [RDX-H]−, however, this ion was not observed in electrospray. A, B, and C show the consecutive and intended mass shift triggered by adducts of Chlorine, Bromine, and Bromo-difluoro-acetate to result in an ion mass well above m/z 300 for the resulting m/z 395.2. A, B, and C also show the different isotopic patterns that can be obtained when using Bromide (1:1) over a Chloride adduct (3:1), improving sensitivity and pattern recognition.

Referring to FIG. 4, three ambient desorption/ionization mass spectra in negative ion mode ESI (A, B, and C) of PETN using: A) Chlorine as an adducting agent at 50 μmol/L; B) Bromine as an adducting agent at 20 μmol/L; and C) Bromo-difluoro-acetate as an adducting agent at 10 μmol/L in solution are shown. Location of the molecular ion was indicated at m/z 315.0 for [PETN-H]− as observed with Chloride and Bromide; however, this ion was not observed above baseline noise when using Bromo-difluoro-acetate, showing a complete conversion to the adduct. A, B, and C also show the different isotopic patterns that can be obtained when using Bromide (1:1) over a Chloride adduct (3:1), improving sensitivity and pattern recognition.

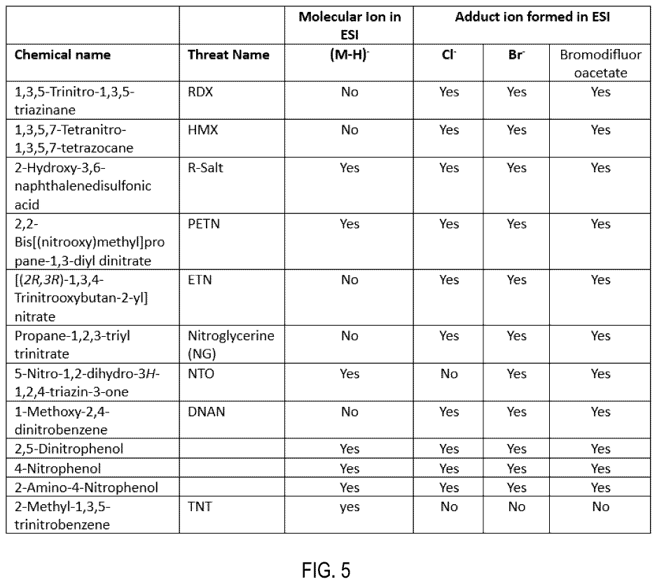

Referring to FIG. 5, a table depicting the threat molecules that, under ESI conditions, form molecular ions (M−H)− or adduct clusters with halogens and Bromo-difluoro-acetate are shown. Various threat molecules were tested in different chemical classes for their ability to form adducts with Bromo-difluoro-acetate and found the adduct formed to be equal or better in MS signal intensity than other typical adducting agents. Thus, this demonstrates that a large number of threats (including those not shown or described herein) can create this adduct with almost 100% completion and are stable enough to be used for mass spectrometric detection.

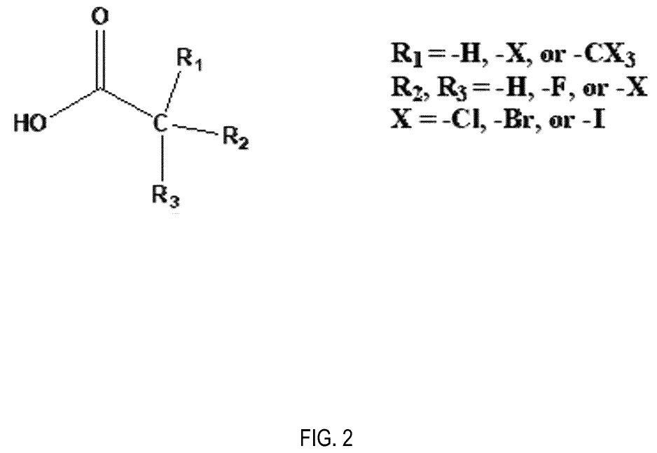

In one embodiment, the adduct agent can be added at a certain concentration to a methanol solution containing the threat to be detected (in solid or dissolved form). In an embodiment, the concentration of the adduct agent added to the solution is between about 1-50 mol/L. In a preferred embodiment, 10 mol/L was used. In an embodiment, the methanol solution is a mixture of methanol and water, and in another embodiment, the solution is a mixture of ethanol and water. The percentage of methanol or ethanol by volume can be between 80-100% in accordance with an embodiment. Then, an electrospray ionization can be applied at a voltage (for example between about 1-4.5 kV, and in a preferred embodiment, 3 kV), and either in solution or during the electrospray process, the adduct is formed and transferred into the gas phase and into the mass spectrometer for analysis. This can be done in the context of high-pressure liquid chromatography (HPLC) by adding the adduct agent at the indicated concentrations to the solvents of the HPLC system and then analyzing an injected sample in the typical fashion (i.e., typical for HPLC, as should be understood by a person of ordinary skill in conjunction with a review of this disclosure) with electrospray ionization mass spectrometric detection at the end. The adduct agent could be added to the solvent stream after the column and as a post column Tee in solvent stream (instead of being present in the HPLC solvent). An adduct agent is considered desirable for this purpose if it forms only that ion in the mass spectrometric analysis (compared to a weak adduct agent only partially forming the ion and leaving some molecular ion present in the MS data). This is demonstrated by FIG. 4, where Bromine for example leaves some M−H ion behind, while Bromo-difluoro-acetate shows only that ion (and no M−H), indicating a stronger binding of Bromo-difluoro-acetate than Bromine. While Bromo-difluoro-acetate is discussed, use of another adduct that similarly only forms an ion in the mass spectrometric analysis is additionally contemplated. An example of a genus adduct with formula F1 is shown in FIG. 2.

In another embodiment, the threat molecule to be detected can be mixed with a solution containing more than one adduct agent. The resulting mass spectrometry data would then indicate which adducts are formed and in what ratio to each other. Ideally, only one adduct would be formed with one agent, indicating its relative binding strength.

In another embodiment, a surface sampling technique can be used where a threat on a surface is collected on a cellulose-based swab for example, and the solvent solution is dripped onto the swab (e.g., about 20 μL). A voltage can then be applied to the swab to create electrospray and analyzed with a mass spectrometer. The adducting agent will be in the solvent added to the swab at the indicated concentration.

Thus, in one example, if a user desires to test for a specific chemical threat (i.e., target analyte), the user can define a specific mass-to-charge (m/z) value or pattern that corresponds to the adducted version of the molecule that the user wants to detect. The chemical threat can be combined with an adducting agent (such as mixing it with a methanol solution containing that adducting agent, such as Bromo-difluoro-acetate) and calculate the expected m/z of the adduct (e.g., target+adduct=adducted ion m/z). Next, if using MS, the user can tell the instrument which m/z peak to look for and with what ratio. For the reasons explained below, Bromo-difluoro-acetate can be a good adducting agent to use to more easily identify whether the chemical threat is present. If those peaks appear, then the target analyte was detected.

In some embodiments, a chemical threat testing kit can be provided. The kit can include a surface testing instrument, such as a surface swab (triangular, square, circular, etc.), an adducting agent, and a solution. The user can then use MS to identify whether the chemical threat being tested is present.

It is intended that the different embodiments, and elements thereof, can be combinable in any way mechanically and chemically possible. While various embodiments have been described and illustrated herein, those of ordinary skill in the art will readily envision a variety of other means and/or structures for performing the function and/or obtaining the results and/or one or more of the advantages described herein. Each of such variations and/or modifications is deemed to be within the scope of the embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize or be able to ascertain using no more than routine experimentation, many equivalents to the specific embodiments described herein. It is, therefore, to be understood that the foregoing embodiments are presented by way of example only and that, within the scope of the appended claims and equivalents thereto, embodiments may be practiced otherwise than as specifically described and claimed. Embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

The above-described embodiments of the described subject matter can be implemented in any of numerous ways. For example, some embodiments may be implemented using hardware, software or a combination thereof. When any aspect of an embodiment is implemented at least in part in software, the software code can be executed on any suitable processor or collection of processors, whether provided in a single device or computer or distributed among multiple devices/computers.

What is claimed is:

1. A method of detecting a target analyte by mass spectrometry, comprising:
contacting a sample suspected of containing the target analyte with a solution comprising Bromo-difluoro-acetate anion (BrCF₂COO—) in an electrospray-compatible solvent;
subjecting the contacted sample to negative-mode electrospray ionization to generate gas-phase ions; and
detecting an adduct ion comprising the target analyte and Bromo-difluoro-acetate, wherein the detected adduct ion exhibits a bromine isotopic doublet having peaks separated by about 2 Daltons and an intensity ratio of about 1:1,
and wherein the mass-to-charge ratio of the adduct ion is shifted relative to the mass-to-charge ratio of the target analyte or, when present, the deprotonated molecular ion of the target analyte.

2. The method of claim 1, wherein the solution comprises Bromo-difluoro-acetate at a concentration of about 10 mol/L.

3. The method of claim 1, wherein the electrospray-compatible solvent comprises methanol.

4. The method of claim 1, wherein the electrospray-compatible solvent comprises ethanol.

5. The method of claim 1, wherein the target analyte is selected from PETN, RD, and nitroglycerin.

6. A method of detecting a chemical threat present on a surface, comprising:
applying a solution comprising an adducting agent to the surface under conditions effective to form, on the surface or in the applied solution, an adduct between the chemical threat and the adducting agent;
transferring at least a portion of the applied solution to an electrospray emitter and applying a capillary voltage to generate gas-phase ions comprising ions of the adduct; and
analyzing the ions with a mass spectrometer to detect the chemical threat;
wherein the adduct has the structure of Formula (I): 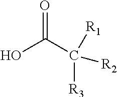
wherein R1 is selected from H, X, and CX3;
wherein R2 is selected from H, F, and X;
wherein R3 is selected from H, F, and X; and
wherein X is selected Cl, Br, and I.

7. The method of claim 6, wherein the solution comprises the adducting agent at a concentration of about 10 mol/L.

8. The method of claim 6, wherein the chemical threat is selected from PETN, RD, and nitroglycerin.

9. A method of detecting a target analyte by mass spectrometry, comprising:
contacting a sample suspected of containing the target analyte with a solution comprising an anionic halogenated carboxylate containing one or more halogen atoms selected from bromine and chlorine;
generating negative-mode electrospray ions;
subjecting the contacted sample to negative-mode electrospray ionization to generate gas-phase ions; and
detecting an adduct ion comprising the target analyte and the halogenated carboxylate, wherein the detected adduct ion exhibits a halogen isotopic pattern characteristic of the one or more halogen atoms present,
and wherein the mass-to-charge ratio of the adduct ion is shifted relative to the mass-to-charge ratio of the target analyte or, when present, the deprotonated molecular ion of the target analyte.

10. The method of claim 9, wherein the halogenated carboxylate is Bromo-difluoro-acetate.

11. The method of claim 9, wherein the halogenated carboxylate comprises bromine and the detected adduct ion exhibits a bromine isotopic doublet with peaks separated by about 2 Daltons and an intensity ratio of 1:1.

12. The method of claim 9, wherein the halogenated carboxylate comprises chlorine and the detected adduct ion exhibits a chlorine isotopic doublet with peaks separated by about 2 Daltons and an intensity ratio of 3:1.

13. The method of claim 9, wherein detecting comprises selected ion monitoring or multiple reaction monitoring of m/z values corresponding to the adduct ion and its isotopologues.

14. The method of claim 9, wherein the solution comprises the anionic halogenated carboxylate at a concentration of about 10 mol/L.

15. The method of claim 9, wherein the target analyte is selected from PETN, RD, and nitroglycerin.
